(19) United States
(12) Patent Application Publication
(10) Pub. No.: US 2024/0341352 A1
(43) Pub. Date: Oct. 17, 2024

(54) NEBULIZER

(71) Applicant: Murata Manufacturing Co., Ltd., Kyoto (JP)

(72) Inventors: Miki IKEDA, Kyoto (JP); Kenghua HSING, Kyoto (JP)

(21) Appl. No.: 18/756,086

(22) Filed: Jun. 27, 2024

Related U.S. Application Data
(63) Continuation of application No. PCT/JP2023/002299, filed on Jan. 25, 2023.

(30) Foreign Application Priority Data
Jan. 26, 2022 (JP) 2022-010230

(51) Int. Cl. A24F 40/10; A24F 40/42; A24F 40/485; A61M 15/00

(52) U.S. Cl. CPC A24F 40/10; A24F 40/42; A24F 40/485; A61M 15/0021

(57) ABSTRACT

A case of a nebulizer includes a discharge outlet that communicates with the outside of the nebulizer. A nozzle has a gas hole and a liquid supply path. The gas hole can supply an output gas. The liquid supply path uses a liquid hole as an opening at one end and a suction inlet as an opening at the other end. The liquid hole is located adjacent to the gas hole and can supply a liquid to an internal space. The suction inlet sucks a liquid to be supplied to the liquid hole. An opening center, which is the centroid of the discharge outlet when an opening surface of the discharge outlet is seen in a plan view, is displaced from a virtual first reference axis. The first reference axis is parallel with the pointing direction of the gas hole and passes through the gas hole.

### CROSS REFERENCE TO RELATED APPLICATION

This is a continuation of International Application No. PCT/JP2023/002299 filed on Jan. 25, 2023, which claims priority from Japanese Patent Application No. 2022-010230 filed on Jan. 26, 2022. The contents of these applications are incorporated herein by reference in their entireties.

### BACKGROUND OF THE DISCLOSURE

### Field of the Disclosure

The present disclosure relates to a nebulizer.

### Description of the Related Art

Patent Document 1 discloses a nebulizer. The nebulizer includes a case having an internal space and a nozzle that atomizes a liquid. The case has an opening for guiding the atomized liquid to the outside of the case. The nozzle is located in the internal space of the case. The nozzle has a gas hole, a liquid hole, and a liquid supply path. The gas hole is used for ejecting an output gas. The liquid hole is used for ejecting a liquid. The liquid supply path is located inside the nozzle. The liquid supply path extends from the liquid hole toward the bottom side of the internal space.
Patent Document 1: Japanese Unexamined Patent Application Publication No. 2011-229709

### BRIEF SUMMARY OF THE DISCLOSURE

It is assumed that a nebulizer, such as that disclosed in Patent Document 1, is used in the orientation in which the pointing direction of the gas hole coincides with the opposite direction of the direction of gravity. Sometimes, however, the nebulizer may be used in the orientation in which the pointing direction of the gas hole is tilted with respect to the axis along the direction of gravity. This makes it difficult to supply a liquid stored in the internal space of the case to the liquid supply path.

To solve the above-described problem, an aspect of the disclosure provides a nebulizer including a case and a nozzle. The case has an inner wall that defines an internal space. The nozzle is located in the internal space and atomizes a liquid stored in the internal space. The case includes a discharge outlet that communicates with outside of the nebulizer. The nozzle has a gas hole and a liquid supply path. The gas hole is able to supply an output gas. The liquid supply path uses a liquid hole as an opening at one end and uses a suction inlet as an opening at the other end. The liquid hole is located adjacent to the gas hole and is able to supply the liquid to the internal space. The suction inlet sucks a liquid to be supplied to the liquid hole. A virtual straight axis which is parallel with a pointing direction of the gas hole and which passes through the gas hole is set to a first reference axis. An opening center, which is a centroid of the discharge outlet when an opening surface of the discharge outlet is seen in a plan view, is displaced from the first reference axis. A direction from the gas hole toward the opening center when the nebulizer is seen in a direction along the first reference axis is set to a specific direction. The suction inlet is entirety located on a side of the specific direction with respect to the gas hole.

It is possible that a user uses the nebulizer by tilting it in the orientation in which the discharge outlet is inclined toward the direction of gravity with respect to the horizontal level. In this case, a liquid stored in the internal space of the case is shifted toward the discharge outlet. With the above-described configuration of the nebulizer, the suction inlet is located on the same side as the opening center. Even if the nebulizer is entirely tilted and the position of a liquid stored in the internal space is shifted, the suction inlet is most likely to remain under the liquid. It is thus unlikely to make it difficult to supply the liquid stored in the internal space to the liquid supply path.

It is less likely that it becomes difficult to supply a liquid stored in the internal space to a liquid hole.

### BRIEF DESCRIPTION OF THE SEVERAL VIEWS OF THE DRAWINGS

FIG. 1 is a perspective view of a nebulizer of an embodiment.

FIG. 2 is an exploded perspective view of the nebulizer of the embodiment.

FIG. 3 is a perspective view of a nozzle and a tank in the embodiment.

FIG. 4 is a sectional view of the nebulizer of the embodiment.

FIG. 5 is a top view of the nebulizer of the embodiment seen through from above.

FIG. 6 is a sectional view of a nebulizer of a modified example.

### DETAILED DESCRIPTION OF THE DISCLOSURE

### Embodiment

An embodiment of a nebulizer will be described below with reference to the drawings. For easy understanding of the embodiment, elements may be shown in enlarged size in the drawings. The dimensional ratio of elements in one drawing may be different from that in another drawing or that of actual elements.

### (Overall Configuration)

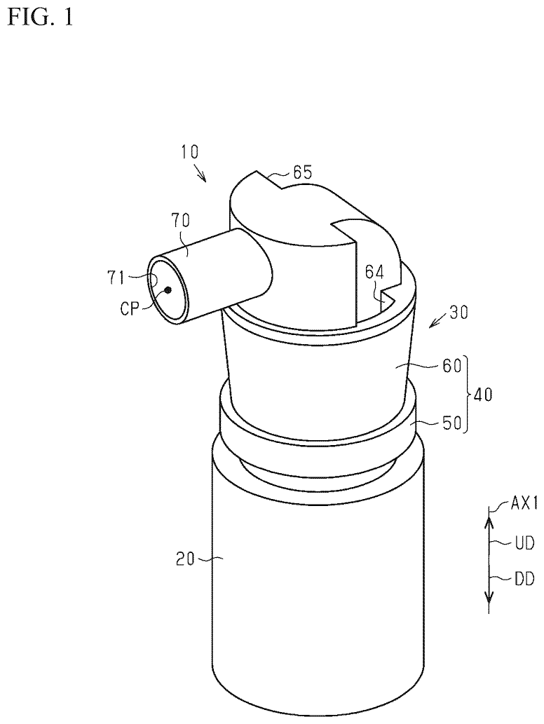

As illustrated in FIG. 1, a nebulizer 10 includes a pump case 20 and a case 30. A gas pumped out of the pump case 20 is supplied to an internal space S of the case 30.

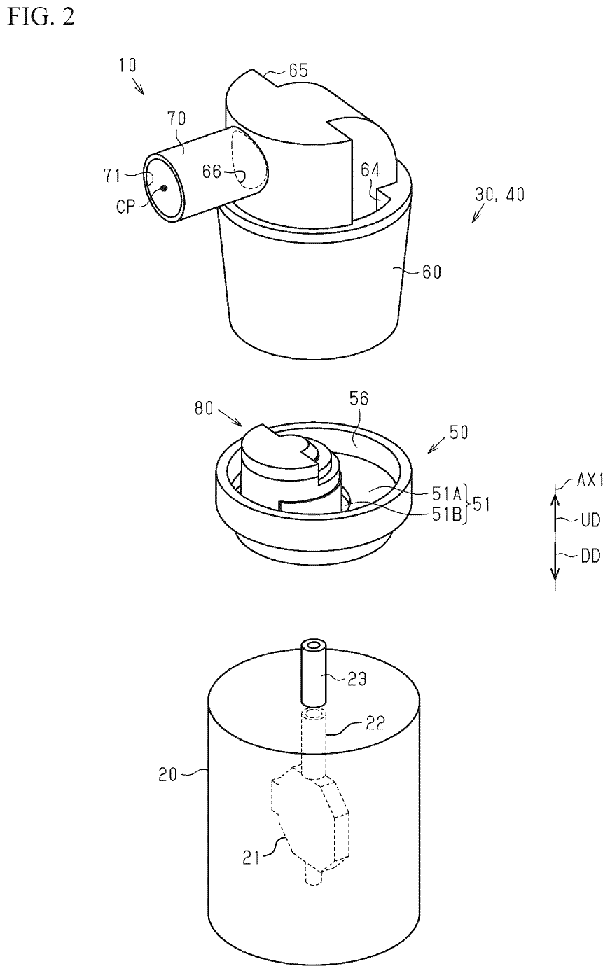

As illustrated in FIG. 2, the nebulizer 10 includes a pump 21 and a tube 22. The pump 21 is located inside the pump case 20. The pump 21 is what is known as a piezoelectric pump. The pump 21 includes a piezoelectric element and a diaphragm. The piezoelectric element is a piezoelectric ceramic element. The diaphragm repeatedly deflects due to the vibration of the piezoelectric element so that the pump 21 can supply air. That is, the pump 21, which is a piezoelectric pump, can pump out a gas.

The tube 22 is connected to the pump 21. Air pumped out of the pump 21 flows inside the tube 22. The pump case 20 includes a connecting pipe 23. The connecting pipe 23 has a tubular shape. The connecting pipe 23 is linked with the tube 22 so that air pumped out of the pump 21 flows out of the connecting pipe 23 of the pump case 20.

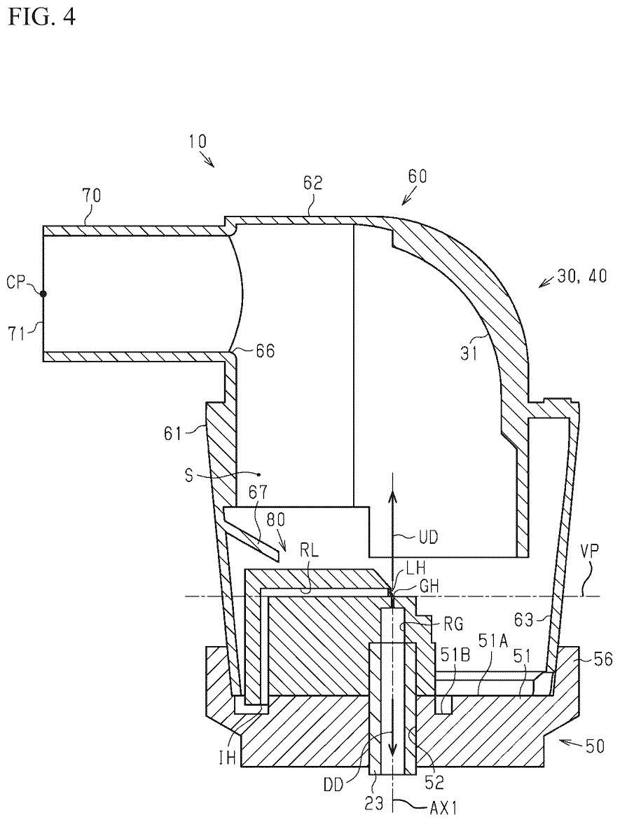

As shown in FIG. 4, the case 30 has an inner wall 31 that defines the internal space S. The case 30 also has a discharge outlet 71 for guiding a liquid atomized in the internal space S to the outside of the case 30. The discharge outlet 71 communicates with the outside of the case 30. Details of the case 30 will be discussed later.

As shown in FIG. 2, the nebulizer 10 includes a nozzle 80. As illustrated in FIG. 4, the nozzle 80 is located in the internal space S of the case 30. The nozzle 80 atomizes a liquid stored in the internal space S.

### (Nozzle)

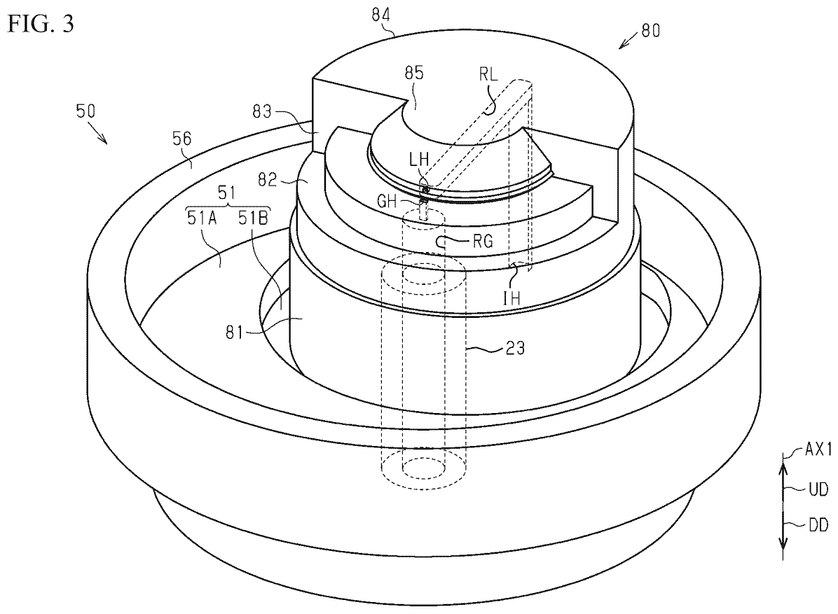

As shown in FIG. 3, the nozzle 80 has a gas supply path RG that links the internal space S and the inside of the connecting pipe 23 with each other. The gas supply path RG is a space for causing a gas pumped out of the pump 21 to flow to the internal space S via the connecting pipe 23. The nozzle 80 has a gas hole GH, which is positioned at an end of the gas supply path RG closer to the internal space S. The gas hole GH is a hole which can supply a gas pumped out of the pump 21 to the internal space S.

It is assumed that the pointing direction of the gas hole GH is an upward direction UD and the opposite direction of the upward direction UD is a downward direction DD. As shown in FIG. 4, a virtual straight axis which is parallel with the upward direction UD and passes through the gas hole GH is set to a first reference axis AX1. The pointing direction of the gas hole GH is a direction determined in the following manner. A viewpoint from which the apparent area of a range surrounded by the outer periphery of the gas hole GH is maximized is first specified. Then, the outer periphery of the gas hole GH is seen from this viewpoint and the centroid of the outer periphery of the gas hole GH is specified. Then, the direction from the specified centroid to the viewpoint is set to the pointing direction of the gas hole GH.

As illustrated in FIG. 3, the nozzle 80 has a liquid supply path RL through which a liquid stored in the internal space S is supplied to the vicinity of the gas hole GH. The nozzle 80 has a suction inlet IH, which is an opening of the liquid supply path RL in the downward direction DD. The nozzle 80 also has a liquid hole LH, which is an opening of the liquid supply path RL in the upward direction UD. That is, the opening at one end of the liquid supply path RL is the liquid hole LH, while the opening at the other end of the liquid supply path RL is the suction inlet IH. The suction inlet IH is an opening which sucks a liquid to be supplied to the liquid hole LH. That is, the liquid supply path RL extends from the liquid hole LH, which ejects a liquid, toward the bottom of the internal space S. The liquid hole LH is located adjacent to the gas hole GH, so that a liquid ejected from the liquid hole LH is supplied toward the gas hole GH. A state in which a liquid is supplied from the liquid hole LH due to a flow of a gas supplied from the gas hole GH is assumed as a state in which the gas hole GH and the liquid hole LH are adjacent to each other.

The nozzle 80 includes a first portion 81 and a second portion 82. The first portion 81 has a cylindrical shape. The second portion 82 has a cylindrical shape. The second portion 82 is disposed on the surface of the first portion 81 which faces the upward direction UD.

The nozzle 80 also includes a third portion 83. The third portion 83 is disposed on the surface of the second portion 82 which faces the upward direction UD. The third portion 83 has a planar shape. When the nozzle 80 is seen from above in the downward direction DD, the third portion 83 covers a semicircular section of the second portion 82 and the center of the second portion 82 and its vicinity. Accordingly, part of the surface of the second portion 82 which faces the upward direction UD is not covered with the third portion 83, that is, it is projected from the third portion 83 and is exposed.

The nozzle 80 also includes a fourth portion 84. The fourth portion 84 is disposed on the surface of the third portion 83 which faces the upward direction UD. The fourth portion 84 has a semicircular planar shape in a plan view. When the nozzle 80 is seen from above in the downward direction DD, the position of the center of the assumed circle of the semicircular fourth portion 84 coincides with that of the center of the circle of the first portion 81. When the nozzle 80 is seen from above in the downward direction DD, the fourth portion 84 covers a semicircular section of the third portion 83.

The nozzle 80 also includes a fifth portion 85. The fifth portion 85 is disposed on the surface of the third portion 83 which faces the upward direction UD. The fifth portion 85 has a semicircular planar shape in a plan view. When the nozzle 80 is seen from above in the downward direction DD, the position of the center of the assumed circle of the semicircular fifth portion 85 coincides with that of the center of the circle of the first portion 81. The diameter of the semicircle of the fifth portion 85 is shorter than that of the fourth portion 84. The fifth portion 85 covers part of the surface of the third portion 83 which faces the upward direction UD. Accordingly, part of the surface of the third portion 83 which faces the upward direction UD is covered with neither the fourth portion 84 nor the fifth portion 85, that is, it is projected from the fourth and fifth portions 84 and 85 and is exposed.

The gas hole GH is opened to the surface of the third portion 83 which faces the upward direction UD. The gas hole GH is located at a portion of this surface which is projected from the fourth and fifth portions 84 and 85. The gas hole GH is located near the side surface of the fifth portion 85.

The liquid hole LH is opened to the side surface of the fifth portion 85. The liquid hole LH is positioned in the vicinity of the gas hole GH. As stated above, the liquid hole LH is located adjacent to the gas hole GH. A liquid discharged from the liquid hole LH is thus supplied toward the gas hole GH.

### (Case)

As illustrated in FIG. 1, the case 30 includes a case body 40 and a pipe 70. As shown in FIG. 4, the case body 40 houses the nozzle 80 therein. The case body 40 includes a tank 50 and a cover 60.

As illustrated in FIG. 2, the tank 50 has a bottom wall 51 and a side wall 56. The bottom wall 51 has a disc-like shape. As shown in FIG. 4, the bottom wall 51 has a first bottom surface 51A and a second bottom surface 51B. The second bottom surface 51B is positioned at a lower level than the first bottom surface 51A in the downward direction DD so as to form a dent. As shown in FIG. 3, the second bottom surface 51B has a substantially ring-like shape as viewed from above in the downward direction DD. The center of the ring-like shape of the second bottom surface 51B is located closer to the discharge outlet 71 than the center of the first bottom surface 51A is. As shown in FIG. 4, the bottom wall 51 has a connecting hole 52 whose flow path has a circular cross section. The connecting hole 52 passes through the bottom wall 51. The connecting hole 52 is located at a position corresponding to the first bottom surface 51A. More specifically, the connecting hole 52 is located in a range surrounded by the second bottom surface 51B. The connecting pipe 23 of the pump case 20 is fitted in the connecting hole 52.

As illustrated in FIG. 2, the side wall 56 protrudes from the outer periphery of the bottom wall 51 toward the opposite direction of the pump 21. The side wall 56 extends from the entirety of the outer periphery of the bottom wall 51. That is, the side wall 56 has a generally tubular shape.

As shown in FIG. 4, the cover 60 has a side wall 61 having a tubular shape as a whole and a top wall 62. The top wall 62 covers the opening of the side wall 61 which faces the upward direction UD. The opening of the side wall 61 which faces the downward direction DD serves as a connection port 63 having a substantially circular shape. The diameter of the connection port 63 substantially coincides with that of the outer periphery of the side wall 56 of the tank 50 which is on the opposite side of the bottom surface 51. The connection port 63 of the cover 60 is connected to the periphery of the side wall 56 of the tank 50 which faces the upward direction UD.

As illustrated in FIG. 2, the cover 60 has a first intake hole 64 and a second intake hole 65. Both of the first and second intake holes 64 and 65 link an internal space and an external space of the cover 60 with each other in the up-down direction.

The cover 60 has an exit 66 that links the internal space and the external space of the cover 60 with each other. The exit 66 is positioned farther in the downward direction DD than the top edge of the side wall 61 of the cover 60. The exit 66 has a circular shape. The pointing direction of the exit 66 is the opposite direction of the pointing direction of the liquid hole LH.

As shown in FIG. 4, the cover 60 has a blocking wall 67. The blocking wall 60 protrudes from the side wall 61 toward the internal space S. The blocking wall 67 extends in a direction intersecting with the first reference axis AX1. The blocking wall 67 is located on the side of the downward direction DD with respect to the discharge outlet 71. The blocking wall 67 extends farther in the downward direction DD as it becomes closer to its forward end. That is, the forward end of the blocking wall 67 is located farther in the downward direction DD than its base end.

The pipe 70 has a tubular shape. A first end of the pipe 70 is linked to the case body 40. More specifically, the first end of the pipe 70 is connected to the exit 66 of the cover 60. The pipe 70 extends in the pointing direction of the exit 66. That is, the pipe 70 extends in a direction perpendicular to the upward direction UD. As shown in FIG. 4, a virtual plane VP perpendicular to the upward direction UD is imagined. In this case, the extending direction of the pipe 70 is parallel with the virtual plane VP.

The pipe 70 has the discharge outlet 71, which is an opening of the pipe 70 on the opposite side of the cover 60. The discharge outlet 71 is an opening for guiding an atomized liquid to the outside of the case 30. That is, a second end of the pipe 70 serves as the discharge outlet 71. The centroid of the discharge outlet 71 when the opening surface of the discharge outlet 71 is seen in a plan view is set to the opening center CP. In the embodiment, the discharge outlet 71 has a circular shape when the opening surface of the discharge outlet 71 is seen in a plan view. Accordingly, the opening center CP is the circle center of the discharge outlet 71.

The internal space S is defined by the inner wall 31 of the case 30 including the inner wall of the tank 50, the inner wall of the cover 60, and the inner wall of the pipe 70. When a user uses the nebulizer 10, part of the nebulizer 10 including the discharge outlet 71 of the pipe 70 is inserted into the mouth of the user.

### (Positional Relationship Between Gas Hole and Opening Center)

As shown in FIG. 4, the opening center CP of the discharge outlet 71 is displaced from the first reference axis AX1.

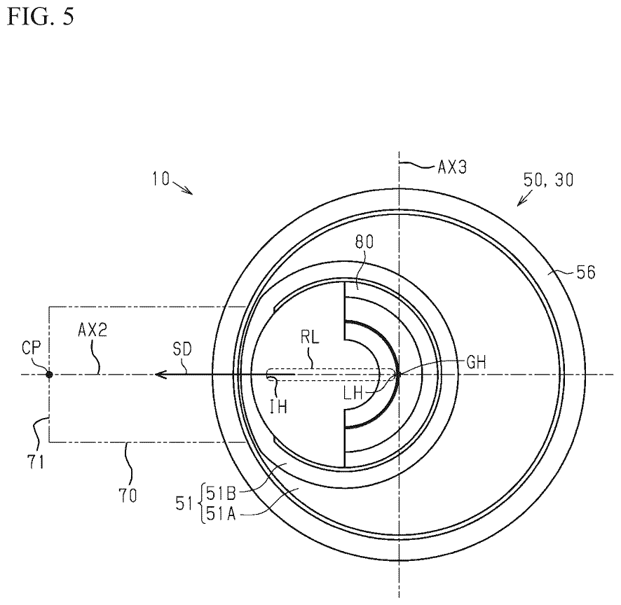

As illustrated in FIG. 5, the direction from the gas hole GH toward the opening center CP when the nebulizer 10 is seen from above in the downward direction DD is set to a specific direction SD. A virtual straight axis which is parallel with the specific direction SD and passes through the gas hole GH is set to a second reference axis AX2. A virtual straight axis which is perpendicular to the second reference axis AX2 and passes through the gas hole GH is set to a third reference axis AX3.

The gas hole GH has a rectangular shape elongated in the direction along the third reference axis AX3 when the nebulizer 10 is seen from above in the downward direction DD. More specifically, the dimension of the long side parallel with the third reference axis AX3 is 0.5 mm, and that of the short side parallel with the second reference axis AX2 is 0.3 mm.

### (Positional Relationship Between Gas Hole and Suction Inlet)

As illustrated in FIG. 5, when the nebulizer 10 is seen from above in the downward direction DD, the greater part of the nozzle 80 is located on the first bottom surface 51A within a range surrounded by the second bottom surface 51B. Nevertheless, as shown in FIG. 4, part of the nozzle 80 extends to the second bottom surface 51B. When the nebulizer 10 is seen from above in the downward direction DD, the suction inlet IH is located within the second bottom surface 51B. The suction inlet IH is positioned inside the dent formed by the second bottom surface 51B. In other words, the suction inlet IH is located in the downward direction DD with respect to the first bottom surface 51A and is also located in the upward direction UD with respect to the second bottom surface 51B.

When the nebulizer 10 is seen from above in the downward direction DD, the entirety of the suction inlet IH is located on the side of the specific direction SD with respect to the gas hole GH. Accordingly, the suction inlet IH is not disposed on the opposite side of the specific direction SD with respect to the gas hole GH. More specifically, as illustrated in FIG. 5, the suction inlet IH is positioned on the side of the specific direction SD with respect to the third reference axis AX3. That is, “the suction inlet IH is located on the side of the specific direction SD with respect to the gas hole GH” includes the meaning, not only that the suction inlet IH is positioned on the second reference axis AX2, but also that the suction inlet IH is not positioned on the second reference axis AX2. It is now assumed that the nebulizer 10 is divided into two regions using the third reference axis AX3 as a boundary when the nebulizer 10 is seen from above in the downward direction DD. In this case, the suction inlet 1H is located in the region containing the opening center CP of the discharge outlet 71. In the embodiment, the suction inlet IH is positioned on the second reference axis AX2.

### (Positional Relationship Between Gas Hole and Blocking Wall)

As shown in FIG. 4, when the case 30 is seen from above in the downward direction DD, the blocking wall 67 is located on the side of the specific direction SD with respect to the gas hole GH. The blocking wall 67 is also located in the upward direction UD with respect to the suction inlet IH. In the embodiment, the blocking wall 67 is located in the upward direction UD with respect to the upper edge of the nozzle 80. When the nebulizer 10 is seen from above in the downward direction DD, the blocking wall 67 extends to overlap the suction inlet IH.

### Operation of Embodiment

A user uses the nebulizer 10 by holding the pump case 20 with the hand, for example, and by inserting part of the nebulizer 10 including the discharge outlet 71 of the pipe 70 into the mouth. In this case, the user most probably uses the nebulizer 10 in the orientation in which the upward direction UD coincides with the opposite direction of the direction of gravity. The nebulizer 10 is used in a state in which a liquid, such as a liquid medicine, is stored in the internal space S. The liquid is thus collected on the bottom wall 51 of the tank 50 within the internal space S.

When the pump 21 is driven, air is pumped out of the pump 21 and flows through the gas supply path RG via the tube 22 and the connecting pipe 23. Air is then ejected from the gas hole GH to the internal space S.

The gas ejected from the gas hole GH passes by the vicinity of the liquid hole LH. This places the vicinity of the liquid hole LH in a negative pressure. A flow is thus generated inside the liquid supply path RL in a direction from the suction inlet IH toward the liquid hole LH. Since the suction inlet IH of the liquid supply path RL is located in the vicinity of the bottom wall 51 of the tank 50, the liquid stored in the tank 50 enters the inside of the liquid supply path RL. Then, the liquid is discharged from the liquid hole LH.

When the liquid discharged from the liquid hole LH is supplied to the vicinity of the gas hole GH, the gas ejected from the gas hole GH collides with the liquid. This changes the liquid into fine droplets. The fine droplets flow through the internal space S toward the discharge outlet 71 due to a flow of the gas and are then ejected from the discharge outlet 71.

Some users may use the nebulizer 10 in the orientation in which the upward direction UD is inclined with respect to the axis along the direction of gravity. For example, if a user uses the nebulizer 10 while lying, the nebulizer 10 is used in such a state. In this case, the discharge outlet 71 is inclined toward the direction of gravity, unlike the nebulizer 10 is used in a state in which the upward direction UD coincides with the opposite direction of the direction of gravity. In this manner, when the nebulizer 10 is inclined with respect to the axis along the direction of gravity, the liquid stored in the internal space S is shifted toward the specific direction SD.

### Advantages of Embodiment

(1) In the embodiment, with respect to the gas hole GH, the suction inlet IH is located on the side of the specific direction SD. That is, with respect to the gas hole GH, the suction inlet IH is located on the same side as the discharge outlet 71. With this configuration, even if the entirety of the nebulizer 10 is tilted and the position of a liquid stored in the internal space S is shifted toward the discharge outlet 71, the suction inlet IH is likely to remain under the liquid. It is thus less likely that it becomes difficult to supply the liquid stored in the internal space S to the liquid supply path RL.
(2) In the embodiment, the forward end of the blocking wall 67 is located farther in the downward direction DD than the base end thereof. With this configuration, even if the entirety of the nebulizer 10 is tilted and the position of a liquid stored in the internal space S farther in the downward direction DD than the blocking wall 67 is shifted toward the discharge outlet 71, the blocking wall 67 is likely to stop the liquid from flowing toward the upward direction UD beyond the blocking wall 67. As a result, the liquid blocked by the blocking wall 67 is likely to remain on the bottom wall 51 of the tank 50. The suction inlet IH is thus likely to remain under the liquid stored on the bottom wall 51 of the tank 50.
(3) In the embodiment, the blocking wall 67 protrudes from the side wall 61 of the cover 60, which is part of the inner wall of the case 30. Hence, the blocking wall 67 can be provided by making a design change to an existing nebulizer without the need to change the outer shape of the case 30.
(4) In the embodiment, some of the atomized droplets may hit the inner wall 31 of the case 30 and be changed to larger droplets. In this case, the larger droplets are moved in the internal space S toward the bottom wall 51 of the tank 50 in the downward direction DD. In the embodiment, the blocking wall 67 extends farther in the downward direction DD as it becomes closer to its forward end. With this configuration, when the larger droplets are recovered from the farther upward direction UD than the blocking wall 67, the blocking wall 67 is less likely to hinder them from moving to the downward direction DD beyond the blocking wall 67. This can enhance the smooth recovery of the droplets.
(5) In the embodiment, the pump 21 is a piezoelectric pump. While the piezoelectric pump is small and lightweight, it is relatively less powerful to pump out a gas. Hence, the vicinity of the liquid hole LH adjacent to the gas hole GH does not sufficiently become in a negative pressure. The force of a flow from the suction inlet IH to the liquid hole LH within the liquid supply path RL accordingly becomes relatively less powerful. As a result, the force of the suction inlet IH to suck a liquid becomes weak, which makes it difficult to supply the liquid to the liquid hole LH via the liquid supply path RL. Given this fact, if the pump 21 is a piezoelectric pump, the above-described advantage (1) can be exhibited more noticeably.
(6) In the embodiment, the extending direction of the pipe 70 is parallel with the virtual plane VP. This can prevent droplets attached to the inner wall 31 of the case 30 from dripping from the discharge outlet 71 of the pipe 70.

### Other Embodiments

The above-described embodiment may be modified in the following manner and be carried out. The embodiment and the following modified examples may be combined with each other and be carried out as long as the resulting configurations do not become technically inconsistent.

The pump 21 is not limited to a piezoelectric pump. For example, the pump 21 may be a rotary pump. Depending on the type of pump 21, the pump case 20 and the connecting pipe 23 may be connected to each other with a hose, for example. In this case, the pump case 20 may be provided separately from the nebulizer 10. That is, the provision of the pump case 20 and the pump 21 for the nebulizer 10 may be omitted. If the nebulizer 10 does not include the pump case 20 and the pump 21, a user can use the nebulizer 10 by holding the tank 50, for example.

The first bottom surface 51A may be tilted farther in the downward direction DD as it becomes closer to the second bottom surface 51B. In this case, a liquid stored in the internal space S is more likely to move to the dent formed by the second bottom surface 51B than the configuration in which the first bottom surface 51A is perpendicular to the first reference axis AX1. The bottom surface of the bottom wall 51 of the tank 50 may be constituted only by the first bottom surface 51A. That is, the provision of the second bottom surface 51B, which is positioned at a lower level than the first bottom surface 51A, may be omitted.

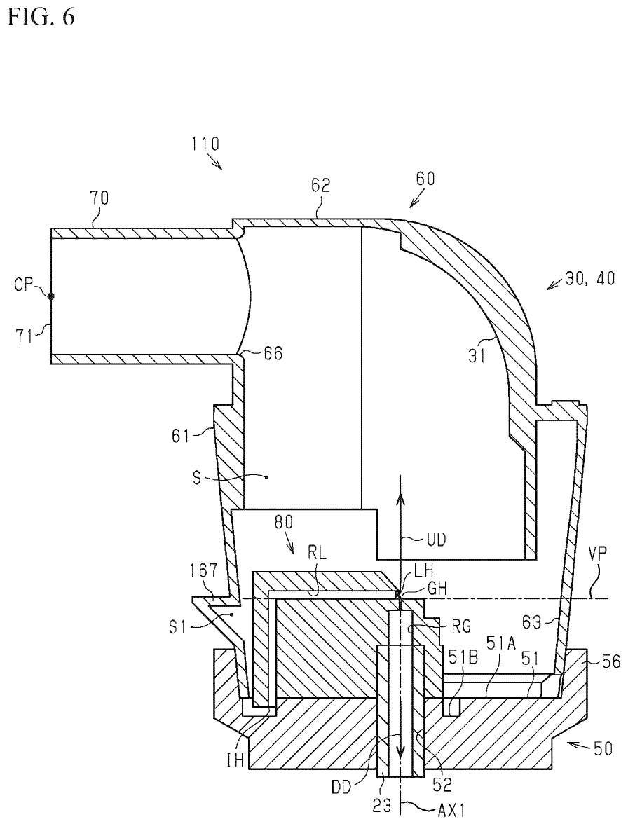

The configuration of the blocking wall 67 is not limited to the example of the embodiment. In a modified example illustrated in FIG. 6, a blocking wall 167 is part of the inner wall 31 of the case 30. “The blocking wall 167 is part of the inner wall 31 of the case 30” refers to a state in which the internal space S is unable to be defined if the blocking wall 167 is removed. In a nebulizer 110 shown in FIG. 6, a recessed portion S1, which serves as a blocking portion located in the internal space S in the downward direction DD with respect to the discharge outlet 71, is swollen in the specific direction D compared with a portion of the internal space S positioned in the upward direction UD with respect to the discharge outlet 71. That is, part of the side wall 61 of the cover 60 is swollen in the specific direction SD. Part of the inner wall 31 that defines the recessed portion S1, which serves as the blocking portion, is used as the blocking wall 167. In this case, too, as in the above-described embodiment, even if the entirety of the nebulizer 110 is tilted, a liquid stored in the internal space S can remain on the bottom wall 51 of the tank 50 because of the blocking wall 167.

In the embodiment, the blocking wall 67 may extend in parallel with the virtual plane VP. Additionally, it is not essential that the blocking wall 67 extends to overlap the suction inlet IH when the nebulizer 10 is seen from above in the downward direction DD.

It is not essential that the extending direction of the pipe 70 is parallel with the virtual plane VP. In one example, the extending direction of the pipe 70 may be inclined in the upward direction UD such that the pipe 70 is positioned farther in the upward direction UD as it becomes closer to the discharge outlet 71. In this case, too, it is possible to prevent droplets attached to the inner wall 31 of the case 30 from dripping from the discharge outlet 71 of the pipe 70.

In another example, the extending direction of the pipe 70 may be inclined in the downward direction DD such that the pipe 70 is positioned farther in the downward direction DD as it becomes closer to the discharge outlet 71. The pipe 70 may extend in the upward direction UD. The extending direction of the pipe 70 may be modified as described above if, at least, the opening center CP of the discharge outlet 71 is displaced from the first reference axis AX1.

It is not essential that the pipe 70 extends straight. For example, the pipe 70 may be curved or may extend so that the sectional area of a flow path is varied. The pipe 70 may be a mask which covers the mouth and the nose of a user. The pipe 70 may be flexible. The provision of the pipe 70 may be omitted. In this case, the exit 66 of the case body 40 serves as the discharge outlet of the case 30.

The configuration of the case 30 is not limited to the example in the embodiment. For example, the case body 40 is constituted by the tank 50 and the cover 60 in the embodiment, but the tank 50 and the cover 60 may be integrated with each other.

The shape of the nozzle 80 is not limited to the example in the embodiment. The shape of the nozzle 80 may be changed suitably in accordance with the shape of the case 30, for example, if the nozzle 80 has the gas hole GH and the liquid hole LH and the entirety of the suction inlet IH is located on the side of the specific direction SD with respect to the gas hole GH.

The shape of the gas hole GH is not limited to a rectangle. The gas hole GH may be circular, elliptical, or square when it is seen from above in the downward direction DD. The shape of the gas hole GH may be a rectangle whose sides parallel with the second reference axis AX2 are long sides.

The suction inlet IH may be located at a position other than on the second reference axis AX2 if it is located on the side of the specific direction SD with respect to the gas hole GH. For example, if plural suction inlets IH are formed because of the provision of plural liquid supply paths RL, all the suction inlets IH are located on the side of the specific direction SD with respect to the gas hole GH. That is, the suction inlets IH may be displaced from the second reference axis AX2 if they are not located on the opposite side of the specific direction SD with respect to the gas hole GH.

A nebulizer, such as that disclosed in Japanese Unexamined Patent Application Publication No. 2011-229709, may sometimes be used in the orientation in which the pointing direction of the gas hole is tilted with respect to the axis along the direction of gravity. In this case, a liquid stored in the internal space of the case may flow out of the discharge outlet.

In the nebulizer 10 of the embodiment, even if the nebulizer 10 is tilted, the blocking wall 67 can stop a liquid stored in the internal space S of the case 30 from flowing until the discharge outlet 71.

In terms of preventing a liquid stored in the internal space S of the case 30 from flowing to the discharge outlet 71, the suction inlet IH may be located at a position other than on the side of the specific direction SD with respect to the gas hole GH. That is, the suction inlet IH may be located on the third reference axis AX3 or on the opposite side of the specific direction SD with respect to the gas hole GH.

The technical concept that can be derived from the above-described embodiment and modified examples is as follows.

### APPENDIX 1

A nebulizer comprising: a case that has an inner wall which defines an internal space; and a nozzle that is located in the internal space and atomizes a liquid stored in the internal space, wherein the case includes a discharge outlet that communicates with outside of the nebulizer, the nozzle has a gas hole and a liquid supply path, the gas hole being able to supply an output gas, the liquid supply path using a liquid hole as an opening at one end and using a suction inlet as an opening at the other end, the liquid hole being located adjacent to the gas hole and being able to supply the liquid to the internal space, the suction inlet sucking a liquid to be supplied to the liquid hole, a virtual straight axis which is parallel with a pointing direction of the gas hole and which passes through the gas hole is set to a first reference axis, and an opening center, which is a centroid of the discharge outlet when an opening surface of the discharge outlet is seen in a plan view, is displaced from the first reference axis, the case also includes a blocking wall which extends in a direction intersecting with the first reference axis, a direction from the gas hole toward the opening center when the nebulizer is seen in a direction along the first reference axis is set to a specific direction, regarding the direction along the first reference axis, an opposite direction of the pointing direction of the gas hole is set to a downward direction, and the blocking wall is located on a side of the downward direction with respect to the discharge outlet and is located on a side of the specific direction with respect to the gas hole when the nebulizer is seen in the direction along the first reference axis.
10, 110 nebulizer
20 pump case
30 case
31 inner wall
40 case body
50 tank
51A first bottom surface
51B second bottom surface
60 cover
67, 167 blocking wall
70 pipe
71 discharge outlet
80 nozzle
AX1 first reference axis
CP opening center
DD downward direction
GH gas hole
IH suction inlet
LH liquid hole
RL liquid supply path
S internal space
S1 recessed portion
SD specific direction
UD upward direction
VP virtual plane

1. A nebulizer comprising:
a case having an inner wall, the inner wall defining an internal space; and
a nozzle located in the internal space and configured to atomize a liquid stored in the internal space, wherein
the case includes a discharge outlet communicating with outside of the nebulizer,
the nozzle has a gas hole and a liquid supply path, the gas hole being configured to supply an output gas, the liquid supply path using a liquid hole as an opening at one end and using a suction inlet as an opening at another end, the liquid hole being located adjacent to the gas hole and being configured to supply the liquid to the internal space, the suction inlet being configured to suck a liquid to be supplied to the liquid hole,
when a virtual straight axis being parallel with a pointing direction of the gas hole and passing through the gas hole is set to a first reference axis, an opening center being a centroid of the discharge outlet when an opening surface of the discharge outlet is seen in a plan view is displaced from the first reference axis, and
when a direction from the gas hole toward the opening center when the nebulizer is seen in a direction along the first reference axis is set to a specific direction, the suction inlet is entirety located on a side of the specific direction with respect to the gas hole.

2. The nebulizer according to claim 1, wherein:
the case includes a blocking wall extending in a direction intersecting with the first reference axis;
regarding the direction along the first reference axis, when an opposite direction of the pointing direction of the gas hole is set to a downward direction, the blocking wall is located on a side of the downward direction with respect to the discharge outlet and is located on the side of the specific direction with respect to the gas hole when the nebulizer is seen in the direction along the first reference axis.

3. The nebulizer according to claim 2, wherein:
the blocking wall protrudes from the inner wall of the case; and
a forward end of the blocking wall is positioned farther in the downward direction than a base end of the blocking wall.

4. The nebulizer according to claim 2, wherein:
a recessed portion positioned in the internal space on the side of the downward direction with respect to the discharge outlet is swollen in the specific direction, compared with a portion of the internal space positioned on a side of the pointing direction of the gas hole with respect to the discharge outlet; and
the blocking wall is a part of the inner wall of the case, the inner wall of the case defining the recessed portion.

5. The nebulizer according to claim 1, wherein:
when an opposite direction of the pointing direction of the gas hole is set to a downward direction, the case has first and second bottom surfaces, the second bottom surface being positioned at a lower level than the first bottom surface in the downward direction so as to form a dent; and
the suction inlet is positioned inside the dent defined by the second bottom surface.

6. The nebulizer according to claim 1, further comprising:
a piezoelectric pump configured to pump out the gas.

7. The nebulizer according to claim 1, wherein:
the case includes a case body and a pipe, the case body housing the nozzle therein, the pipe having a tubular shape, a first end of the pipe being linked with the case body, a second end of the pipe being used as the discharge outlet;
when the pointing direction of the gas hole is set to an upward direction and a virtual plane perpendicular to the upward direction is imagined, an extending direction of the pipe is parallel with the virtual plane or is inclined in the upward direction such that the pipe is positioned farther in the upward direction as the pipe becomes closer to the discharge outlet.

8. The nebulizer according to claim 2, wherein:
when an opposite direction of the pointing direction of the gas hole is set to a downward direction, the case has first and second bottom surfaces, the second bottom surface being positioned at a lower level than the first bottom surface in the downward direction so as to form a dent; and
the suction inlet is positioned inside the dent defined by the second bottom surface.

9. The nebulizer according to claim 3, wherein:
when an opposite direction of the pointing direction of the gas hole is set to a downward direction, the case has first and second bottom surfaces, the second bottom surface being positioned at a lower level than the first bottom surface in the downward direction so as to form a dent; and
the suction inlet is positioned inside the dent defined by the second bottom surface.

10. The nebulizer according to claim 4, wherein:
when an opposite direction of the pointing direction of the gas hole is set to a downward direction, the case has first and second bottom surfaces, the second bottom surface being positioned at a lower level than the first bottom surface in the downward direction so as to form a dent; and
the suction inlet is positioned inside the dent defined by the second bottom surface.

11. The nebulizer according to claim 2, further comprising:
a piezoelectric pump configured to pump out the gas.

12. The nebulizer according to claim 3, further comprising:
a piezoelectric pump configured to pump out the gas.

13. The nebulizer according to claim 4, further comprising:
a piezoelectric pump configured to pump out the gas.

14. The nebulizer according to claim 5, further comprising:
a piezoelectric pump configured to pump out the gas.

15. The nebulizer according to claim 2, wherein:
the case includes a case body and a pipe, the case body housing the nozzle therein, the pipe having a tubular shape, a first end of the pipe being linked with the case body, a second end of the pipe being used as the discharge outlet;
when the pointing direction of the gas hole is set to an upward direction and a virtual plane perpendicular to the upward direction is imagined, an extending direction of the pipe is parallel with the virtual plane or is inclined in the upward direction such that the pipe is positioned farther in the upward direction as the pipe becomes closer to the discharge outlet.

16. The nebulizer according to claim 3, wherein:
the case includes a case body and a pipe, the case body housing the nozzle therein, the pipe having a tubular shape, a first end of the pipe being linked with the case body, a second end of the pipe being used as the discharge outlet;
when the pointing direction of the gas hole is set to an upward direction and a virtual plane perpendicular to the upward direction is imagined, an extending direction of the pipe is parallel with the virtual plane or is inclined in the upward direction such that the pipe is positioned farther in the upward direction as the pipe becomes closer to the discharge outlet.

17. The nebulizer according to claim 4, wherein:
the case includes a case body and a pipe, the case body housing the nozzle therein, the pipe having a tubular shape, a first end of the pipe being linked with the case body, a second end of the pipe being used as the discharge outlet;
when the pointing direction of the gas hole is set to an upward direction and a virtual plane perpendicular to the upward direction is imagined, an extending direction of the pipe is parallel with the virtual plane or is inclined in the upward direction such that the pipe is positioned farther in the upward direction as the pipe becomes closer to the discharge outlet.

18. The nebulizer according to claim 5, wherein:
the case includes a case body and a pipe, the case body housing the nozzle therein, the pipe having a tubular shape, a first end of the pipe being linked with the case body, a second end of the pipe being used as the discharge outlet;
when the pointing direction of the gas hole is set to an upward direction and a virtual plane perpendicular to the upward direction is imagined, an extending direction of the pipe is parallel with the virtual plane or is inclined in the upward direction such that the pipe is positioned farther in the upward direction as the pipe becomes closer to the discharge outlet.

19. The nebulizer according to claim 6, wherein:
the case includes a case body and a pipe, the case body housing the nozzle therein, the pipe having a tubular shape, a first end of the pipe being linked with the case body, a second end of the pipe being used as the discharge outlet;
when the pointing direction of the gas hole is set to an upward direction and a virtual plane perpendicular to the upward direction is imagined, an extending direction of the pipe is parallel with the virtual plane or is inclined in the upward direction such that the pipe is positioned farther in the upward direction as the pipe becomes closer to the discharge outlet.
